(19) United States
(12) Patent Application Publication
(10) Pub. No.: US 2025/0145995 A1
(43) Pub. Date: May 8, 2025

(54) MULTI-FUNCTIONAL COMBINATION OUTLINE TOOL

(71) Applicants: GUO HONG LIANG, San Francisco, CA (US); Richard Liang, San Francisco, CA (US); Edwin Liang, San Francisco, CA (US)

(72) Inventors: GUO HONG LIANG, San Francisco, CA (US); Richard Liang, San Francisco, CA (US); Edwin Liang, San Francisco, CA (US)

(21) Appl. No.: 18/942,730

(22) Filed: Nov. 10, 2024

Related U.S. Application Data
(63) Continuation of application No. 13/638,891, filed on Jan. 17, 2013, now Pat. No. 9,102,938.

(51) Int. Cl. C12N 15/113; A61K 48/00; C07H 19/06; C07H 19/067; C07H 19/16

(52) U.S. Cl. CPC C12N 15/113; A61K 48/00; C07H 19/06; C07H 19/067; C07H 19/16

(57) ABSTRACT

The present invention is a set of Multi-functional Combination Outline Tools, used to measure the shape of vertical objects and copy their shapes on flat materials. The multi-functional combination outline tool a detection recorder as the main body and three auxiliary bases to form three independent measuring tools. It can measure the shapes of flat surfaces, angled planes, curved surfaces, serrated surfaces, wavy surfaces, and other object surfaces, and the shapes of elliptical objects (such as toilet bases) and circular objects (such as cylinders). It is used in the measurement of wood floorings installation.

### RELATED APPLICATION

The inventor has submitted and filed with US Patent Bureau an application on Apr. 25, 2024 for “THREE-IN-ONE MULTIFUNCTIONAL WOOD FLOORING OUTLINE TOOL”, and claims U.S. Provisional Patent No. 36/638,891. This application is still in progress, and the title for the invention is now changed to: “MULTI-FUNCTIONAL COMBINATION OUTLINE TOOL”

### FIELD OF THE INVENTION

The present invention relates to a measuring tool for construction installation engineering, which is specially designed for the installation of wood floorings and can also be used for the installation and measurement of flat materials such as tiles, marble, wood boards, and metal plates.

### BACKGROUND OF THE INVENTION

As an important building material, wood floorings are widely used in house construction and decoration projects. In addition to traditional hardwood floors, many new wood floorings made using new materials and technologies have been produced, such as laminate flooring, vinyl flooring, etc. These new wood floorings have the advantages of being beautiful and durable, light in weight, easy to install, and widely used.

During the installation of wood flooring, various measurement problems are bound to occur. For example, if the walls in the space of a building are not perpendicular, parallel, or have irregular shapes, which do not match the standard specifications of the wood flooring, or if there are obstacles such as toilets, cylinders, etc., it is necessary to measure and cut the wood flooring to specification.

The shape of wood floorings is generally rectangular, and wood floorings panels are connected by a tongue and grove locking system. Therefore, wood floorings must be laid in a single direction and extended in sequence. When encountering obstacles that require cutting to specification, they can only be accurately measured and cut on the side of the extension of the laid wood flooring for proper and tight connection between the wood floorings that also perfectly match the shape of the obstacles. Therefore, there are high requirements for accurate measurements.

Currently, most of the tools used for measuring wood floorings rely on the operator's feeling to judge the accuracy of the measurement and lack a reliable reference basis. Based on these problems often encountered in wood flooring installation work, a set of multi-functional measuring tools is designed to help meet the accurate measurement needs in daily work.

Basing on the principle of parallel lines, a straight line that is fixed perpendicularly to two parallel lines will have identical trajectories along the line. It will maintain the same trajectory when contacting and moving along either a regular or irregular surface.

According to this principle, the multi-functional combination outline tool uses the wood flooring or the contacted object as a reference and utilizes a simple mechanical structure to maintain perpendicularity between the measuring tool and reference object. The resultant recorded trajectory is congruent to the trajectory of the detector movement.

### SUMMARY OF THE INVENTION

The multi-functional combination outline tool bases on a detection recorder and configured to three different measuring functions according to the three bases. In the first combination and the second combination, the detection recorder moves accordingly during measurement and detects the change in the trajectory of the contacted object, which is recorded by the marker.

The first combination of the multi-functional combination outline tool, hereinafter referred to as “Combination A” consists of a detection recorder and a front base mechanically connected to maintain perpendicularity to each other. When measuring with wood flooring as a reference, the front base slides on the edge of the wood flooring with the vertical edge of the front base stabilizing movement.

The second combination of the multi-functional combined outline tool, hereinafter referred to as “combination B” consists of a detection recorder and a rectangular base, which further consists of a front base and a rear base. When measuring a toilet or a cylinder, the rectangular base is clamped on both sides of the wood flooring and fixed, so that the detection recorder can slide and measure stably.

The third combination of the multi-functional combined outline tool, hereinafter referred to as “Combination C” consists of a detection recorder, a front base, and a rear base. The dual detection wheels are used as detectors to roll on the smooth surface of the object, so that the marker pen can stably record its trajectory.

In Combination A and Combination B, the detector holder, configured by the detection recorder, can be equipped with different detectors accordingly for different objects, as well as adjusted for height as needed.

The detection recorder configures a marker pen holder that moves within a sliding groove of the detection recorder main board, allowing for adjustments of the recording range as needed.

### DESCRIPTION OF THE DRAWINGS

As the specification of the present invention proceeds and is described in detail in conjunction with the accompanying drawings, the functions, purposes, and advantages of the present invention will be demonstrated to those skilled in the field. In the accompanying drawings, the same reference numerals always represent the same components.

FIG. 1 is an exploded view of the detection recorder.

FIG. 2 is a side plan view of the main board of the detection recorder.

FIG. 3 is an exploded perspective view of the detection recorder main board, the detector holder and marker holder.

FIG. 4 is a top view of the detector wheel.

FIG. 5 is a side view of the detector rod.

FIG. 6 is a side view of the long detector rod.

FIG. 7 is a perspective view of the slide guide bracket.

FIG. 8 is a side view of the slide guide bracket.

FIG. 9 is a top view of the slide guide bracket.

FIG. 10 is a perspective view of the detection recorder.

FIG. 11 is an exploded view of the multi-functional combination outline tool combination “A”.

FIG. 12 is a perspective view of the front base of Combination A.

FIG. 13 is a side view of the front base of Combination A.

FIG. 14 is a top view of the front base of Combination A.

FIG. 15 is a perspective view of Combination A.

FIG. 16 is a perspective view of the bottom of Combination A.

FIG. 17 is a perspective view of the front base of Combination A.

FIG. 18 is a perspective view of Combination A.

FIG. 19 is a top view of Combination A.

FIG. 20 is a top view of Combination A.

FIG. 21 is a right-side view of Combination A.

FIG. 22 is a left-side view of Combination A.

FIG. 23 is a front view of Combination A.

FIG. 24 is a rear view of Combination A.

FIG. 25 is a bottom view of Combination A.

FIG. 26 is an implementation reference diagram of Combination A.

FIG. 27 is an implementation reference diagram of Combination A.

FIG. 28 is an implementation reference diagram of Combination A.

FIG. 29 is an exploded view of multi-functional combined outline tool combination “B”.

FIG. 30 is a perspective view of the rear base of Combination B.

FIG. 31 is an exploded perspective view of the combined connecting link of Combination B.

FIG. 32 is a perspective view of Combination B.

FIG. 33 is a perspective view of Combination B.

FIG. 34 is an exploded view of an extended version, Combination “B2”.

FIG. 35 is a perspective view of the combined connecting link of Combination B2.

FIG. 36 is a perspective view of Combination B2.

FIG. 37 is a perspective view of the bottom of Combination B2.

FIG. 38 is a top view of Combination B2.

FIG. 39 is a right-side view of Combination B2.

FIG. 40 is a left-side view of Combination B2.

FIG. 41 is a front view of Combination B2.

FIG. 42 is a rear view of Combination B2.

FIG. 43 is a bottom view of Combination B2.

FIG. 44 is an implementation reference diagram of Combination B.

FIG. 45 is an implementation reference diagram (1) of Combination B.

FIG. 46 is an implementation reference diagram (2) of Combination B.

FIG. 47 is an implementation reference diagram (3) of Combination B.

FIG. 48 is an exploded view of the multi-functional combination outline tool Combination “C”.

FIG. 49 is an exploded perspective view of the front base of Combination C.

FIG. 50 is an exploded perspective view of the rear base of Combination C.

FIG. 51 is a top view of the detection wheel of Combination C.

FIG. 52 is a perspective view of Combination C.

FIG. 53 is a perspective view of Combination C.

FIG. 54 is a perspective view of the bottom of Combination C.

FIG. 55 is a top view of Combination C.

FIG. 56 is a right-side view of Combination C.

FIG. 57 is a left-side view of Combination C.

FIG. 58 is a front view of Combination C.

FIG. 59 is a rear view of Combination C.

FIG. 60 is a bottom view of Combination C.

FIG. 61 is an implementation reference diagram of Combination C.

FIG. 62 is an implementation reference diagram of Combination C.

FIG. 63 is an implementation reference diagram of Combination C.

FIG. 64 is an implementation reference diagram of Combination C.

FIG. 65 is a perspective view of Combination C with an extended marker holder.

FIG. 66 is a perspective view of Combination C with an extended marker holder.

FIG. 67 is a top view of the Combination C with extended marker holder.

FIG. 68 is an implementation reference diagram of Combination C with extended marker holder.

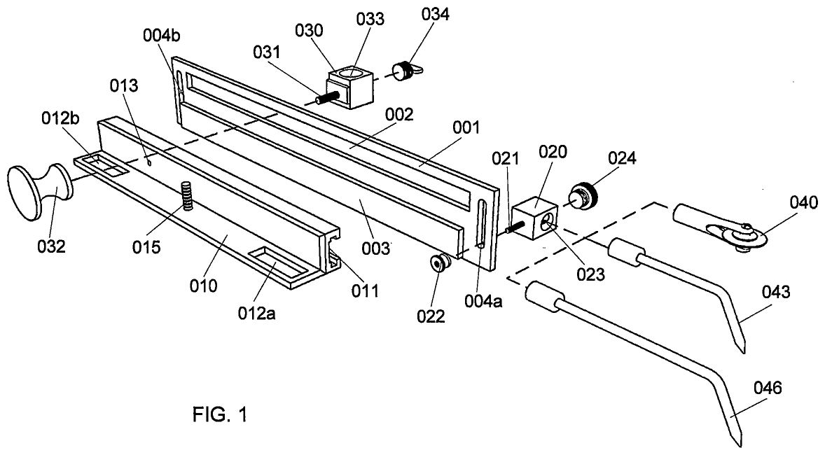

FIG. 1 shows the composition of the detection recorder including a main board 001, a detector holder 020 and its respective fixing nut 022 and fastening screw 024, a marker holder 030 and its respective fixing nut 032 and fastening screw 034, a slide guide bracket 010, and detector recorder configured by a detector wheel 040, a detector rod 043, and a long detector rod 046.

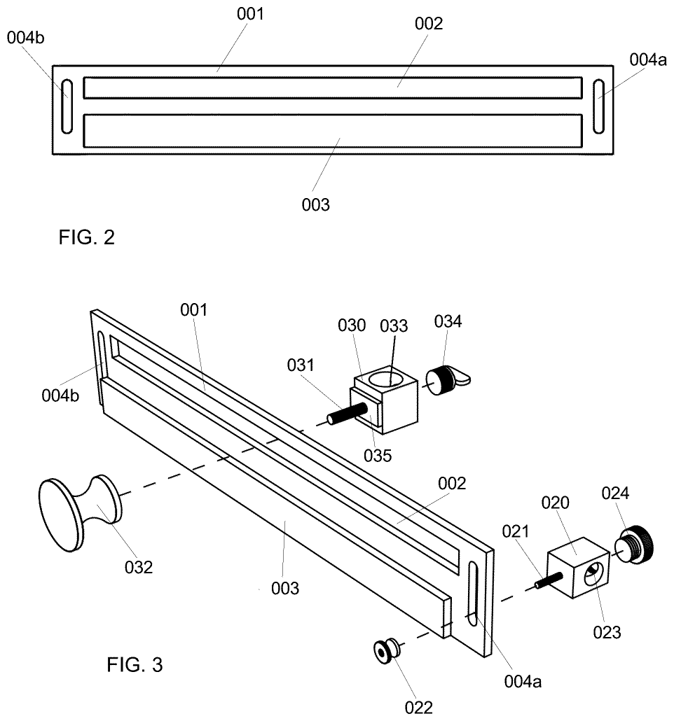

FIG. 2 shows identically sized vertical slots 004a and 004b, symmetrically positioned at each end of the main board 001 for the detector holder. Both ends of the main board can be used to hold detectors according to the orientation of the detector recorder.

FIG. 3 shows the screw 021 of the detector holder secured with nut 022. The height of screw 021 can be adjusted vertically in the slot 004a.

FIG. 3 also shows the marker holder 030, which has a side convex width the same as the width of the groove 002, and the marker holder is limited to horizontal movement within the groove and secured with knobs 032 to a specific position.

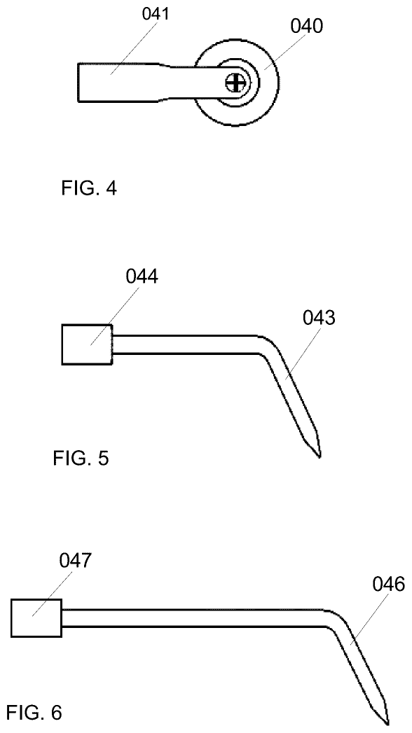

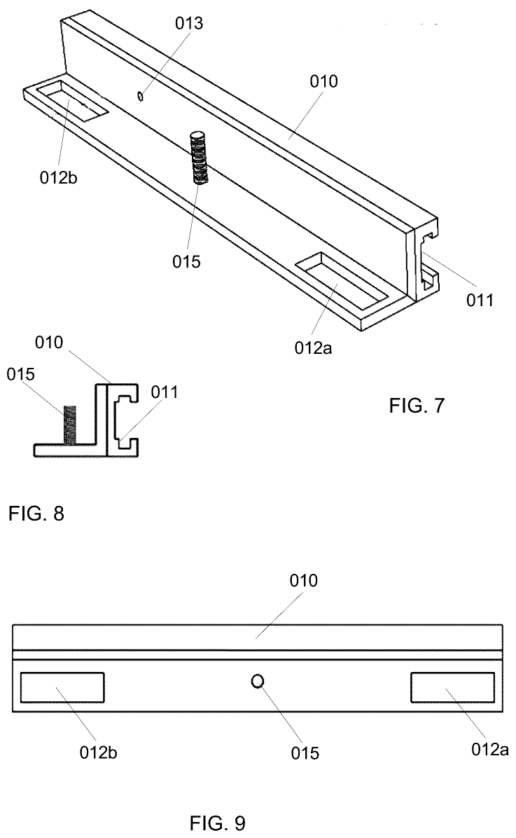

FIGS. 4, 5, and 6 show the detector wheel 040, the detector rod 043, and the long detector rod 046. The diameters of their support rods 041, 044, and 047, are the same as the diameter of the detector mounting hole 023.

FIG. 8 shows the rail groove 011 of the slide guide bracket 010, allowing free movement of the slide bar 003 of the main board 001.

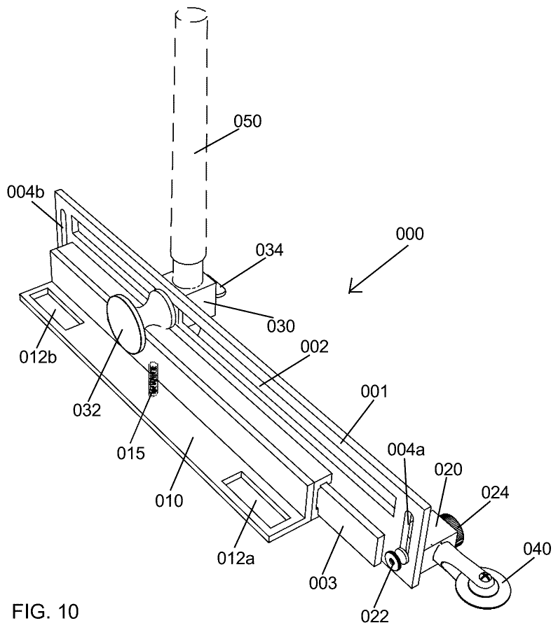

FIG. 9 shows the slide guide bracket 010 with identically sized rectangular holes 012a and 012b symmetrically positioned at each end to be fitted with the rectangular blocks of the bases according to the combination.

FIG. 10 shows the detection recorder equipped with a marker pen 050 (indicated by a dotted line). When measuring, the main board 001 installed on the slide guide bracket 010 can move forward and backward. The detector at the front end of the main board 001 moves back and forth according to the shape of the object, and the marker pen at the back synchronously records the movement trajectory of the detector. The detection recorder is designated number 000.

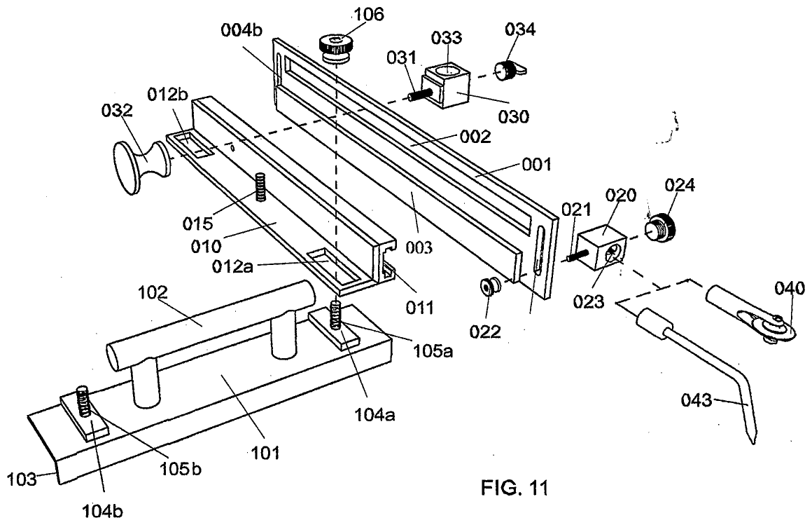

FIG. 11 is an exploded view of the multi-functional combination outline tool combination A, comprising the detection recorder 000 and the front base 101. The overall is designated number 100.

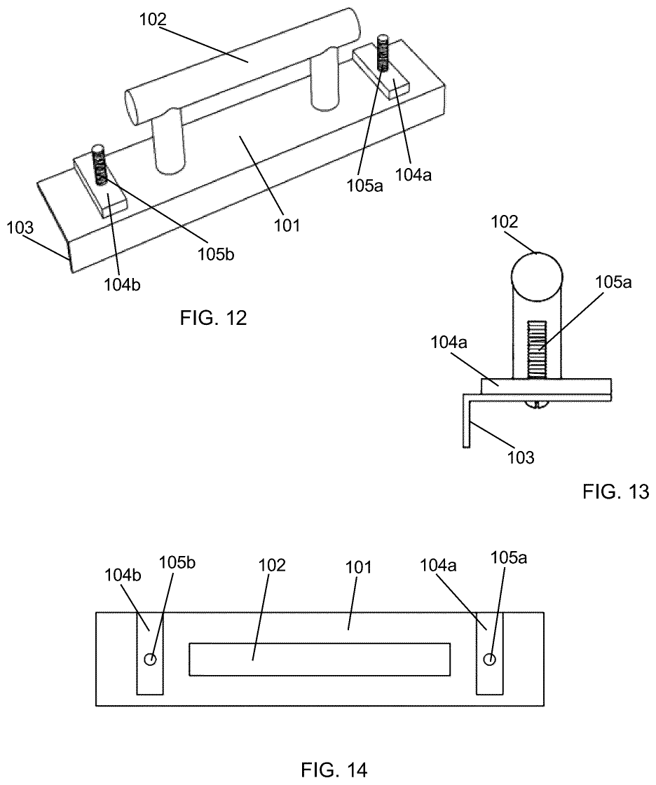

FIG. 12 shows the operating handle 102, rectangular blocks, 104a and 104b, and the screws, 105a and 105b, of the front base 101.

FIG. 13 shows the vertical edge 103 perpendicular to the front base 101 that limits movement along the edge of a wood flooring during measurement.

FIG. 14 shows identical rectangular blocks, 104a and 104b, symmetrically positioned on each side of the top of the front base 101 fitted to dimensionally identical rectangular holes, 012a and 012b, of the detection recorder slide guide bracket.

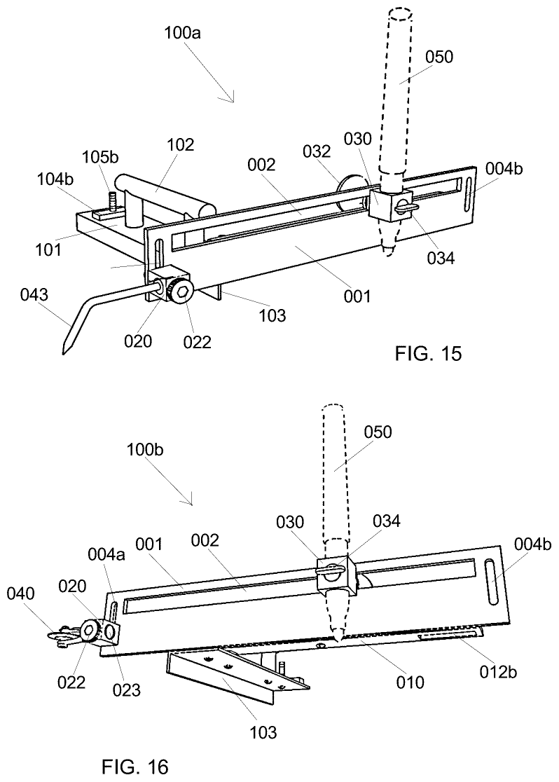

FIG. 15 is combination A 100 fitted with detector rod 043, which is designated as “100a”.

FIG. 16 is combination A 100 fitted with detector wheel 040, which is designated as “100b”.

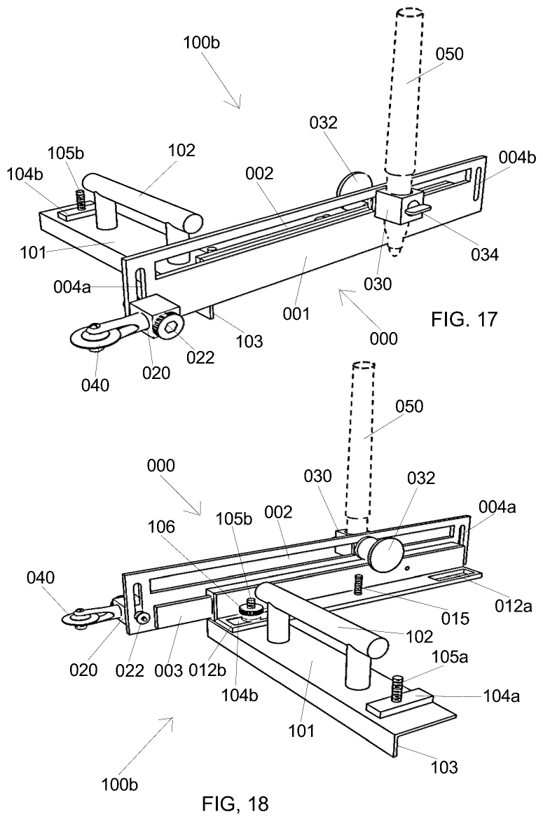

FIG. 17 and FIG. 18 show the detection recorder 000 able to be mounted on either side of the front base 101, which can be adjusted per the operator's handedness.

FIG. 17 and FIG. 18 show the functionality of the combination A in the measurement of a wall. In reaching the end of the wall, an unmeasured portion remains due to obstruction of the front base 101. The detection recorder 000 can be switched to the other side of the front base 101, and the unmeasured portion can be recorded continuing along the previous track line.

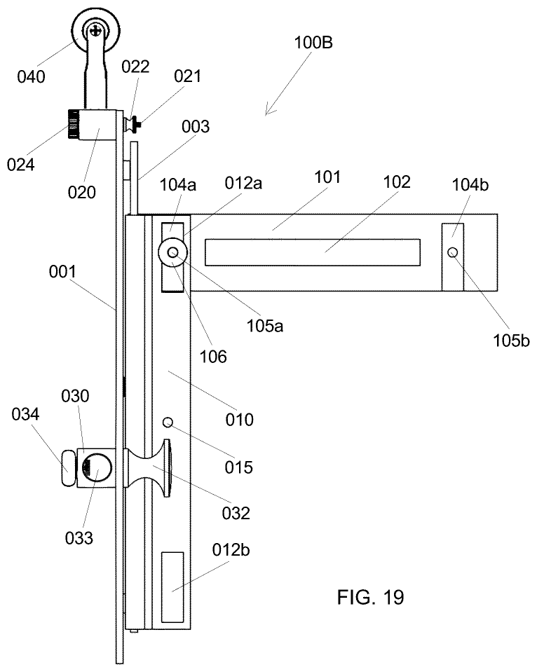

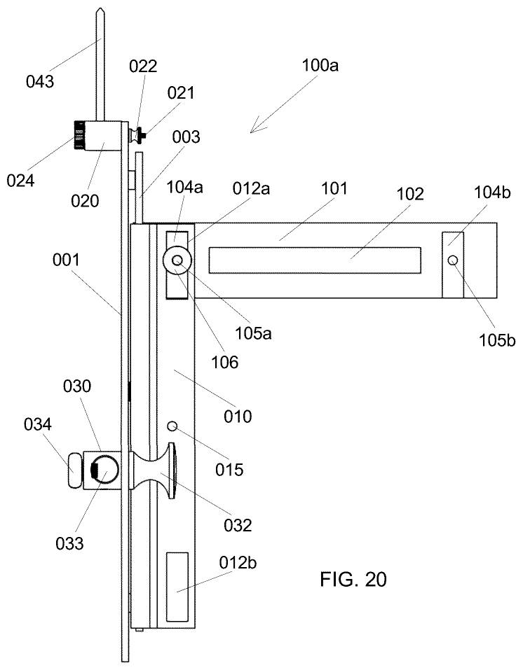

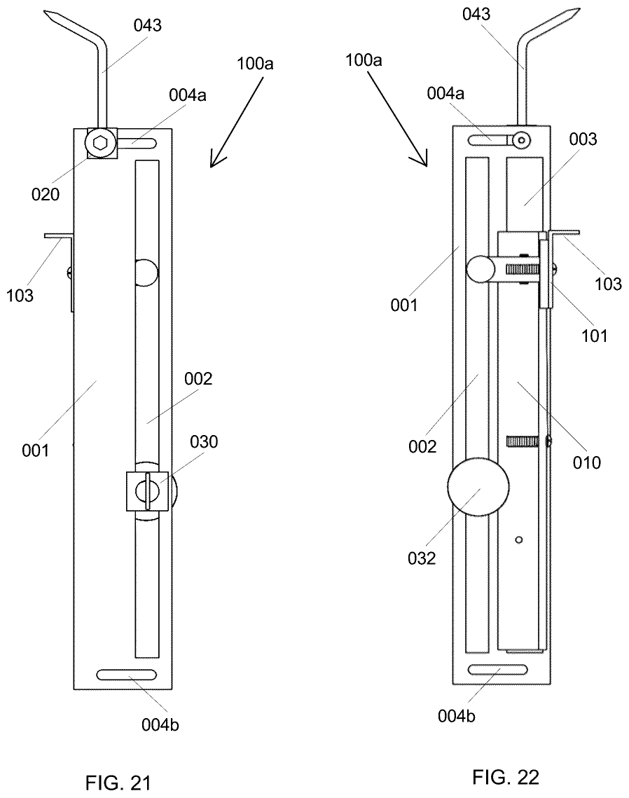

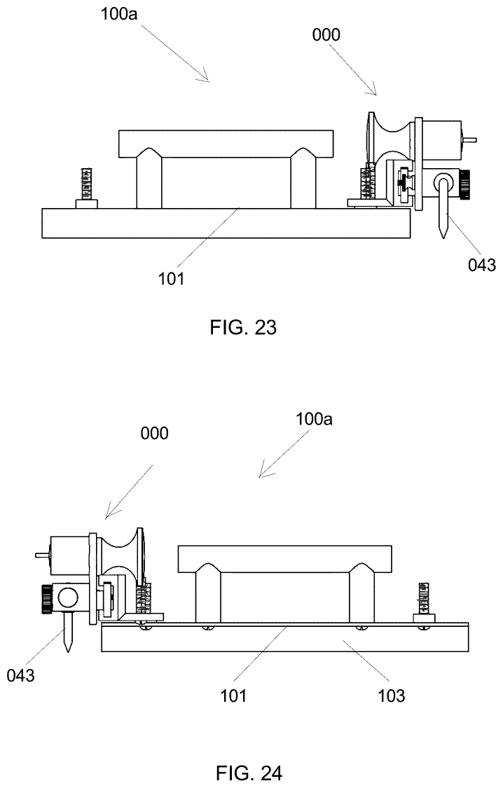

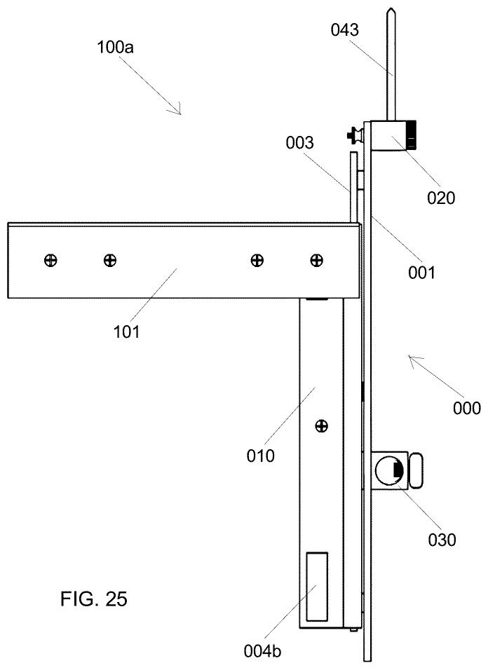

FIG. 19 shows the detection recorder 000 perpendicular to the front base 101 through the tight fit of rectangular block 104a to the rectangular hole 012a, such that the centerline joining the center points of the detector and marker pen, and the wood flooring remains perpendicular as the tool in combination A is moved along the edge of the wood flooring.

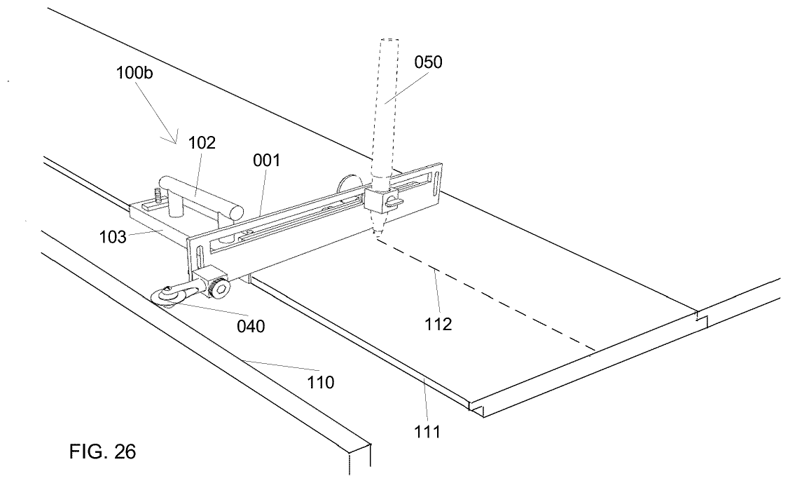

FIG. 26 is an implementation reference diagram of combination A 100b, holding the handle 102 of the front base 101 with the right hand (not shown) and the main board 001 of the detection recorder with the left hand (not shown). The vertical edge 103 of the front base is moved along edge 111 of the wood flooring, and the detector wheel 040 detects the contour of the wall 110, with the marker pen 050 synchronously recording the trajectory 112.

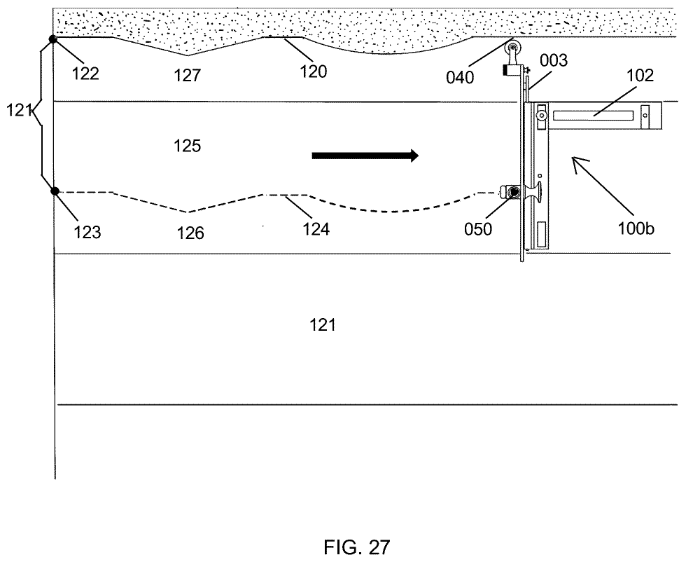

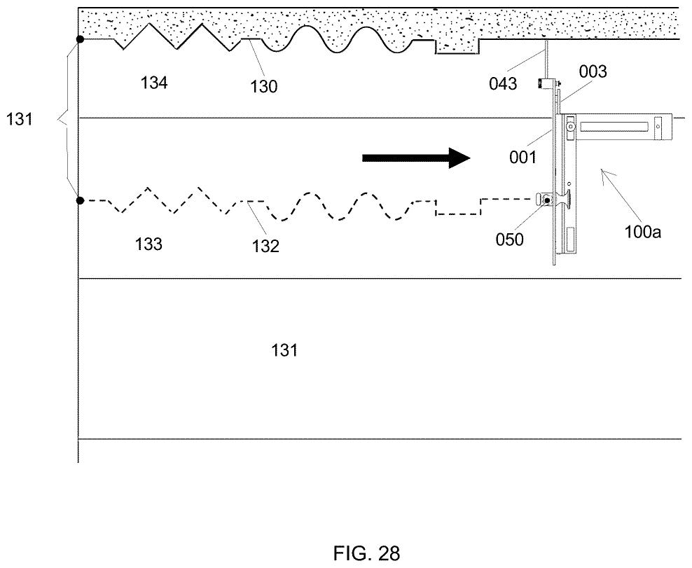

FIG. 27 is an implementation reference diagram of Combination A 100b measuring a wall 120 with planar, beveled, and curved surfaces in a space 127. As shown in the figure, the width of the wood flooring 121 is of standard size, and the distance 121 from the detection start point 122 to the recording start point 123 is measured. When the measurement is completed, the portion 125 above the recorded contour line 124 is removed and discarded, while the portion 126 below the contour line 124 has the same contour and dimensions as in space 127.

Continuing with the implementation reference diagram in FIG. 27, after the measurement is completed, the traced wood flooring is removed and cut. Then a complete wood flooring plank is re-laid in its original position, and the cut portion of the wood flooring 126 is connected to the new wood flooring, covering the space 127.

FIG. 28 is an implementation reference diagram of Combination A 100a for measurement of a wall 130 with flat, jagged, and wavy surfaces. The detector rod 043 moves along the surface of the wall 130 and the marker 050 records the contour 132 of the wall 130 on the wood flooring 131. The portion 133 below the contour line 132 have the same contour and dimensions as in space 134. The operation and principle are the same as described in FIG. 27.

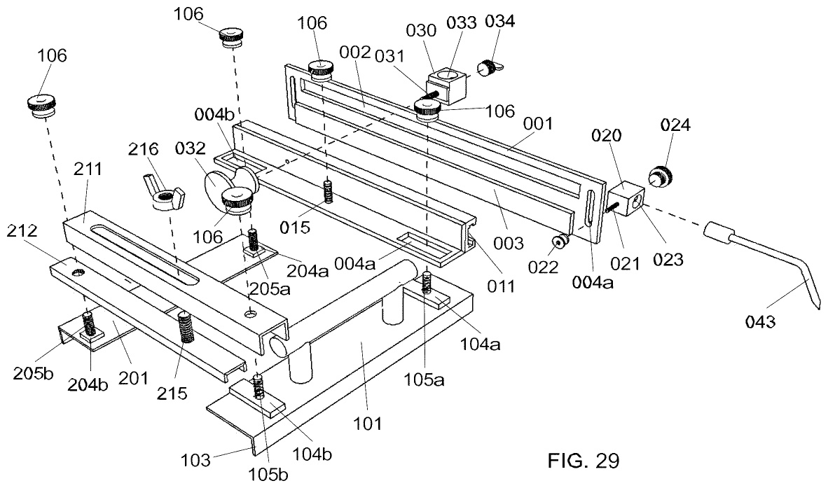

FIG. 29 is an exploded view of a multi-functional combination outline tool combination B, which comprises of a detection recorder 000 combined with front base 101, rear base 201, and combined connecting link 210. The overall designation is 200a.

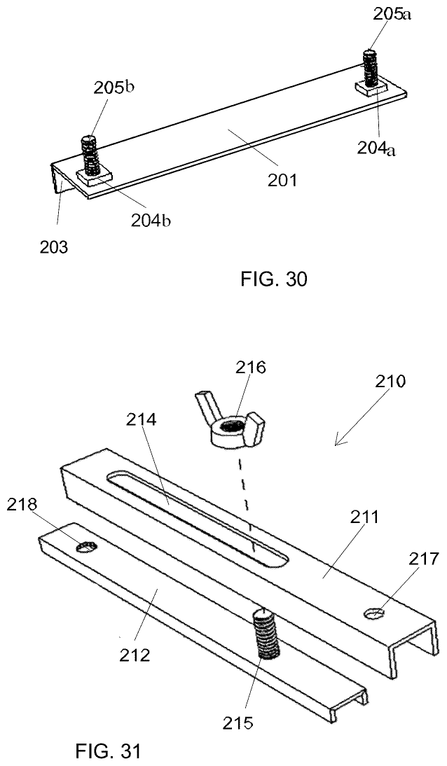

FIG. 30 shows symmetrically positioned, identically sized square blocks 204a and 204b and screws 205a and 205b on each side of the top of rear base 201.

FIG. 31 shows a combined connecting link 210 comprised of a grooved connecting link 211 and a grooved sub-connecting link 212. which the latter's length can be telescopically adjustable within the female link. The two links are linked by screw 215 passing through groove 214 and secured with butterfly nut 216. A hole 217 in the grooved connecting link is for screw 105 of the front base 101 to pass through and is used to connect the connecting link and front base. The hole 218 in the grooved sub-connecting link 212 is for screw 205 of the rear base 201 to pass through and is used to connect the sub connecting link and rear base.

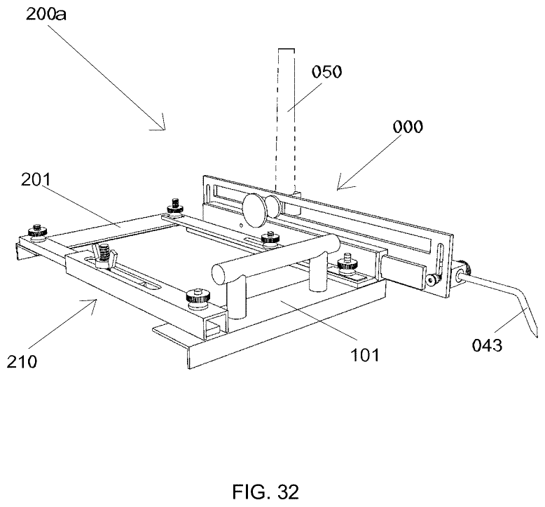

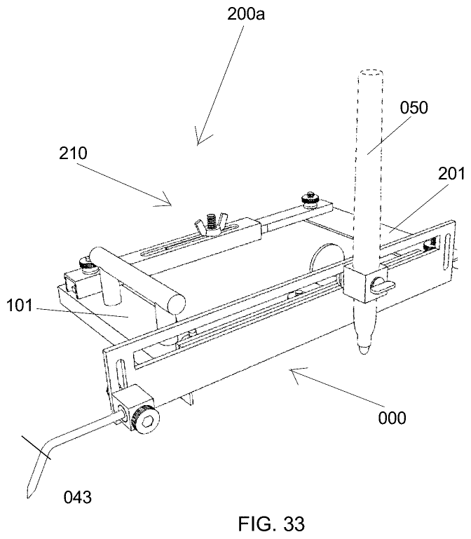

FIG. 32 shows the rectangular base of combination B 200 consisting of the slide guide bracket 010 of the detection recorder and the front base 101, the rear base 201, and the combined connecting link 210.

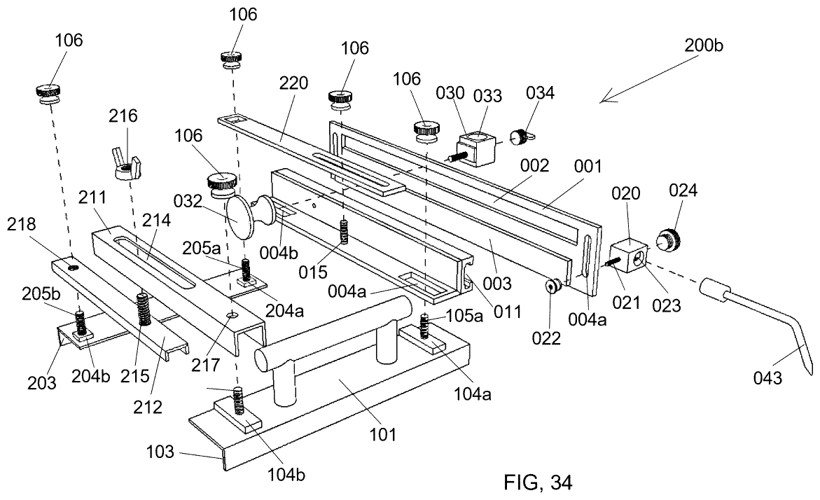

FIG. 34 is an exploded view of combination B 200 with a widened base and an adjustable connecting link 220 between the slide guide bracket 010 and the rear base 201. The overall designation is 200b.

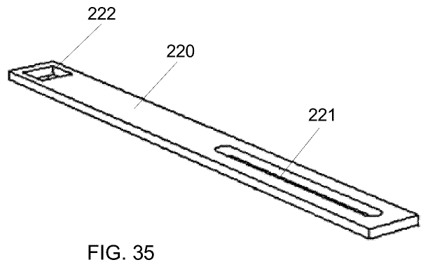

FIG. 35 shows the connecting link 220, and its slot 221 and square hole 222. Square hole 222 has identical dimensions as square blocks 204a and 204b of the rear base.

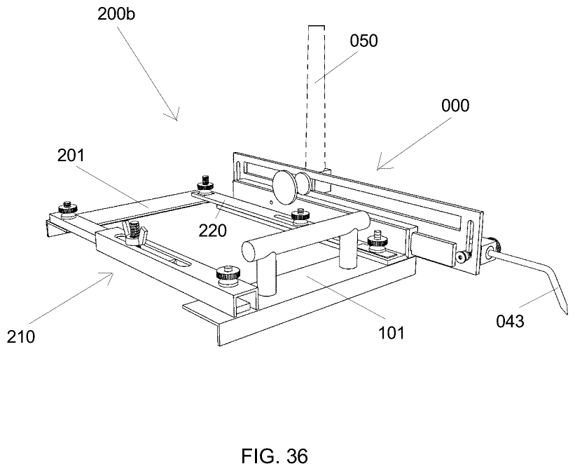

FIG. 36 shows combination B 200b, consisting of the rectangular base, slide guide bracket 010 of the detection recorder, the connecting link 220, and the front base 101, the rear base 201, and the combined connecting link 210.

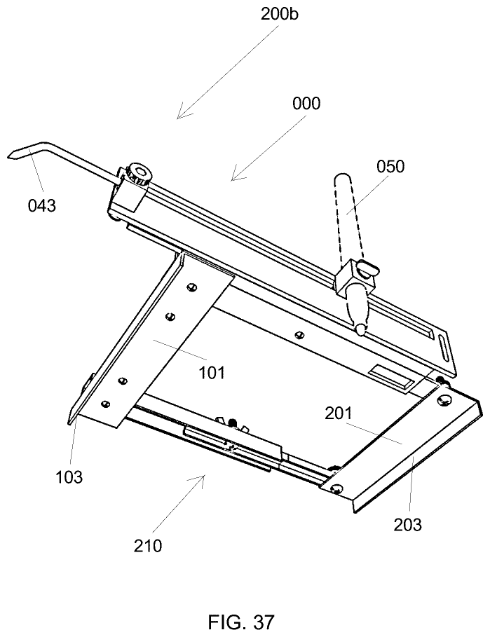

FIG. 37 shows the rectangular base of combination B 200b. The vertical edge 103 of the front base and the vertical edge 203 of the rear base are clamped to both sides of the wood flooring to positioning gliding. The width of the rectangular base can be adjusted by the combination of the connecting links 210 and 220, suiting various sizes of commonly available flooring.

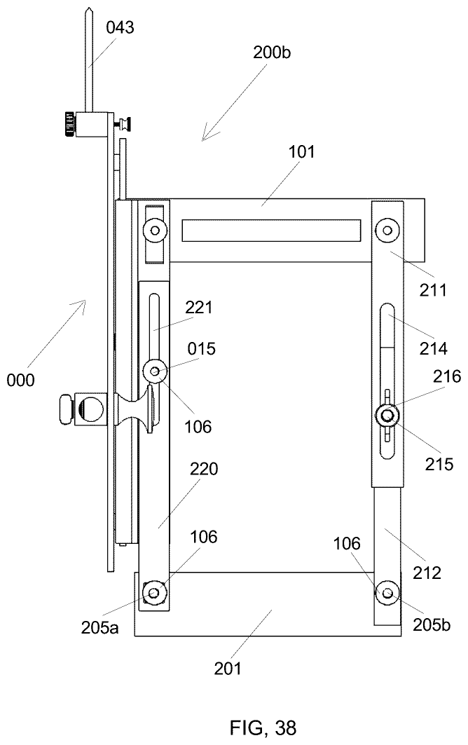

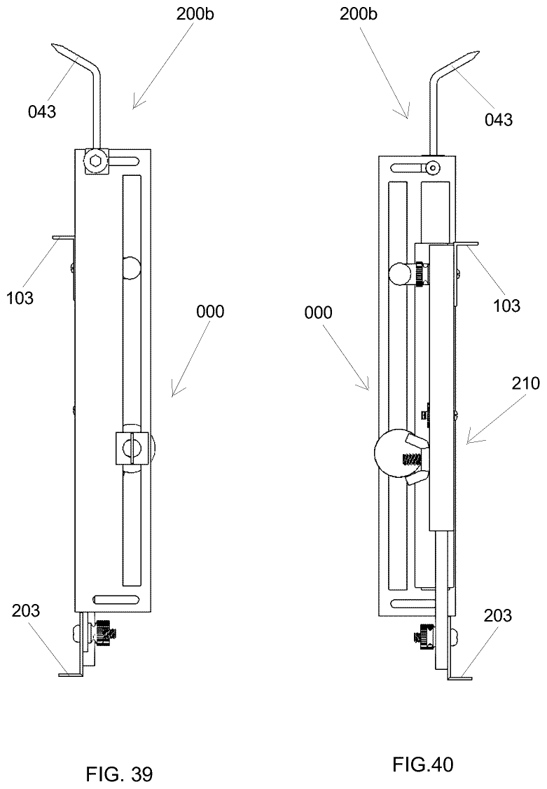

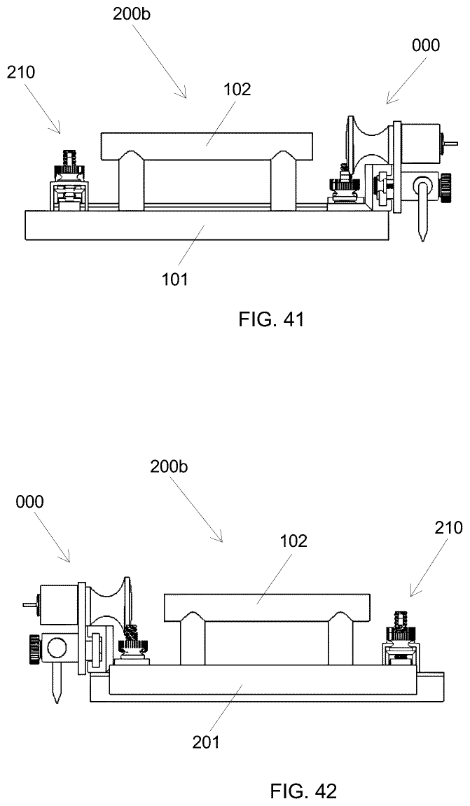

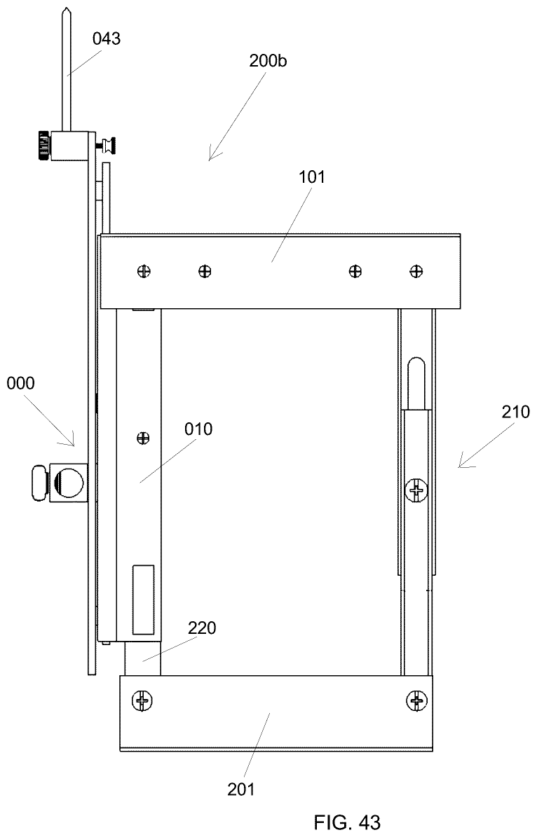

FIG. 38 is a top view of combination B 200b, showing the front base 101 and the rear base 201 each joined perpendicularly to the detection recorder 000 through connecting fasteners.

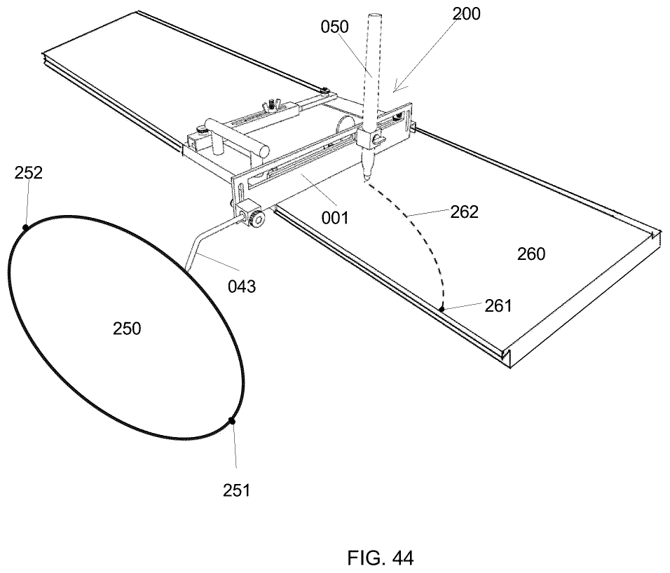

FIG. 44 is an implementation reference diagram of Combination B 200 tracing an oval, representative of a toilet base. As shown, the rectangular base of Combination B 200 is clamped on both sides of the wood flooring. The tip of the detector rod 043 contacts and begins detecting at center point 251 with marker pen 050 positioned at point 261 on the wood flooring plank. Using two hands (not shown) to secure the detection recorder, track the oval toilet base from one center point 251 to the other center point 252 by contacting the detector rod 043 along the base, while the marker pen 050 simultaneously records the contour 262 on the wood flooring 260.

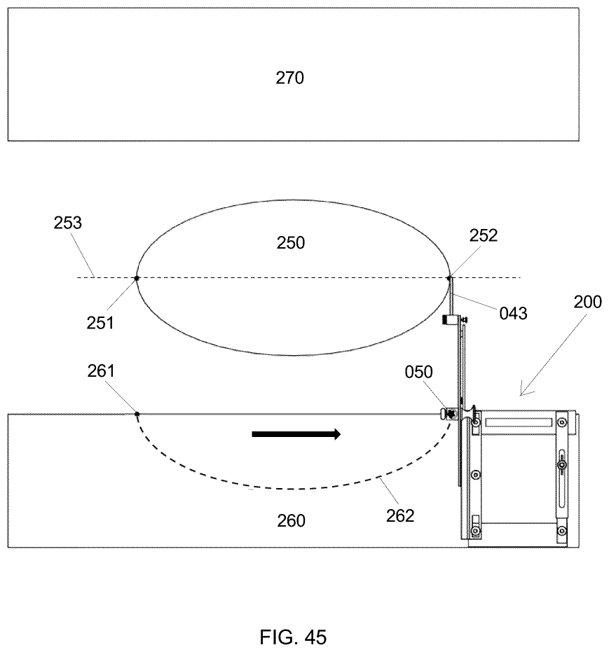

FIG. 45 shows that combination B 200 utilizing a stepwise method for tracing the overall outline of the object, which first traces each half the oval toilet base separately, then combining the two flooring pieces.

FIG. 45 is an implementation reference diagram of the combination B 200 for a method in tracing the oval toilet base 250. The two ends of the wood floorings 260 and 270 are first aligned with each other and parallel to the central axis 253 of the oval. Combination B 200 is clamped to the wood flooring, where the detector rod 043 touches the center point 251 of the toilet base as the starting point. The marker is fixed at the edge at position 261 of the wood flooring as the starting point. When the detector rod 043 moves from center point 251 along the toilet base to the other center point 252, the marker 050 simultaneously records the contour 262 of half of the toilet base on the wood flooring 260.

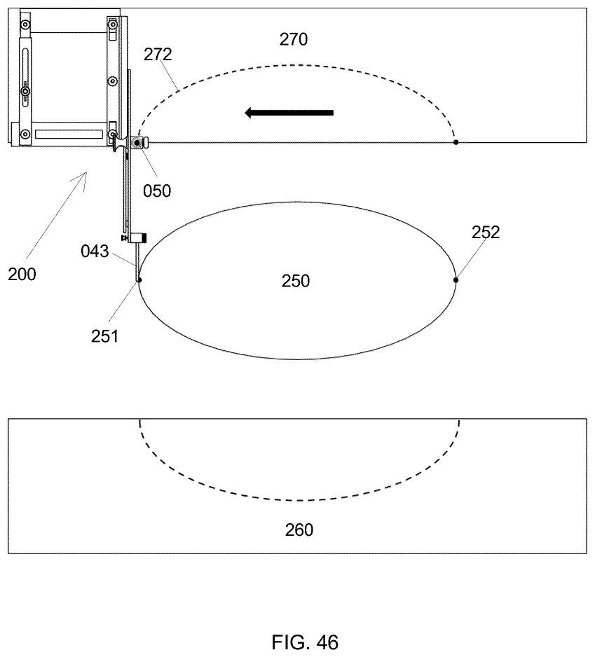

FIG. 46 is the implementation reference diagram of step 2 of tracing flooring for a toilet base with combination B 200. Repeat the operation process of FIG. 45. Move the detector rod 043 from center point 252 along the toilet base to the other center point 251, which the marker pen 050 simultaneously records the contour 272 of the other half of the toilet base on the wood flooring 270.

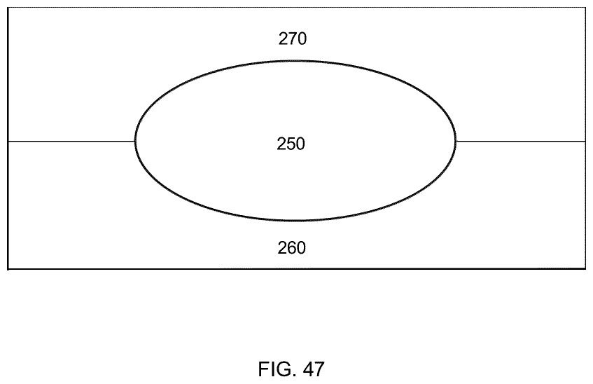

FIG. 47 is a finished view of the cut wood flooring 260 and 270 joined and installed on the toilet base 250.

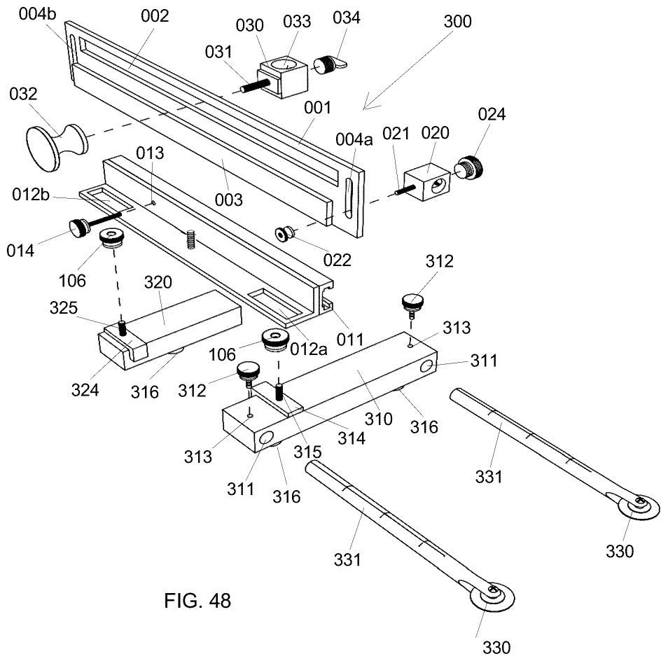

FIG. 48 is an exploded view of the multi-functional contour tracing tool Combination “C”, comprising a detection recorder 000, a front base 310, a rear base 320, and dual detector wheels 330. The overall designation is 300.

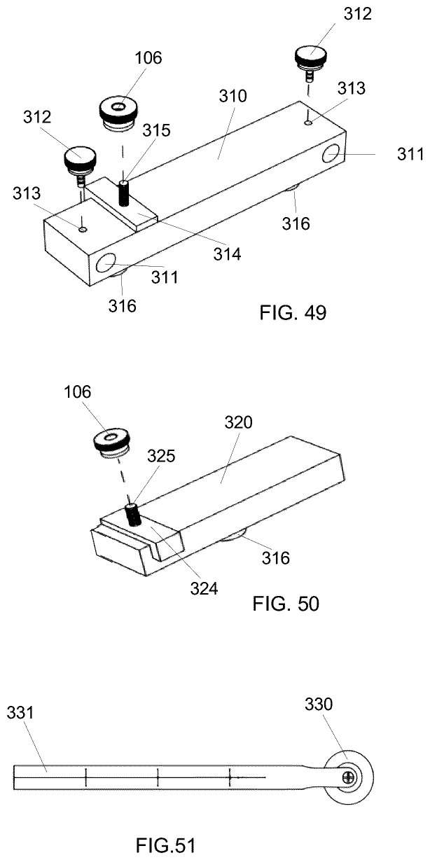

FIG. 49 shows the front base 310, with the rectangular block 314 on the top, which is to be fitted with the rectangular hole 012a of the detection recorder sliding guide bracket 010; both of identical dimensions. The vertical surfaces of the front base have circular mounting holes 311 with same diameter to load the detector wheel 330; the screw holes 313 and screws 312 on the top are used to secure the detector wheel in place.

FIG. 50 shows the rear base 320 and the rectangular block 324 at the top to be fitted with the rectangular hole 012b of the detection recorder slide guide bracket 010. Both are of identical shape and dimension.

FIG. 51 shows a top view of the long detector wheel 330 with the support rod 331 having the same diameter as the front base mounting hole 311.

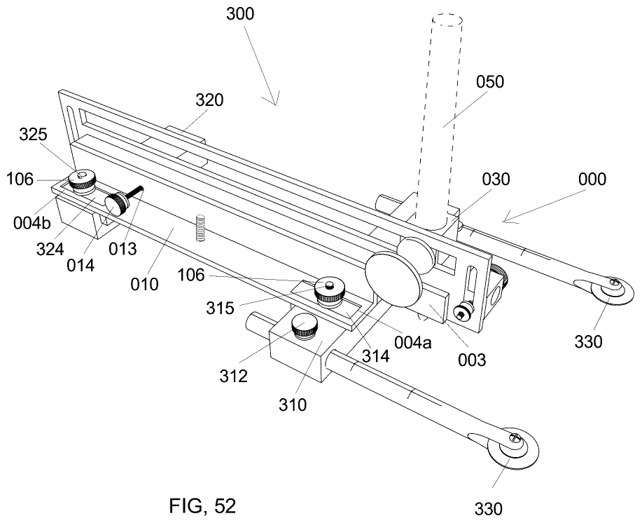

FIG. 52 shows the detector recorder 000 combined with the front base 310 and the rear base 320, secured with a screw nut 106. The detection recorder main board 001 is secured by screw 014 tightened in the screw holes 013 of the slide guide bracket 010, which secures the main board within the bracket. The dual detector wheels 330 are secured by screws 312 at the top of the front base 310.

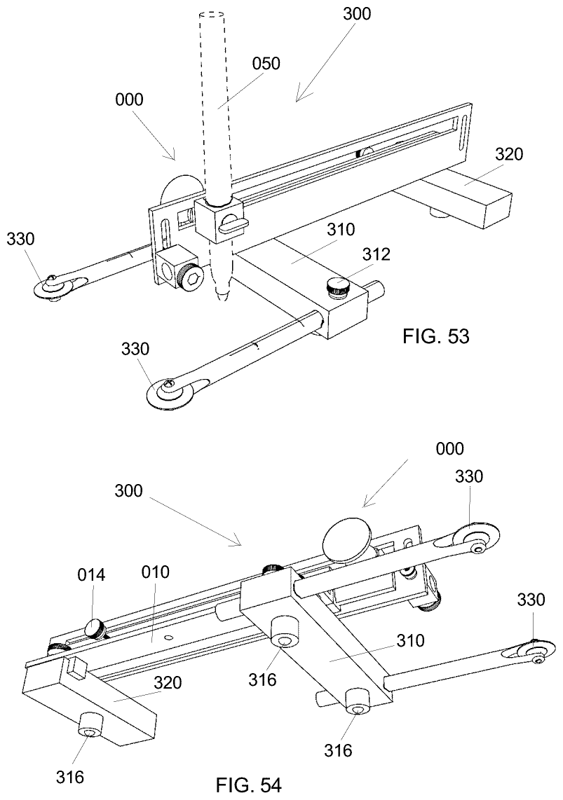

FIG. 53 shows the marker pen 050 fixed in a specific position at the main board 001. The length of the dual detector wheels 330 can be adjusted according to the distance of the target surface. This can be achieved by adjusting the position of the marker pen holder 030 or moving the main board 001 and locking the position of the marker 050 with screw 014 at the desired start point.

FIG. 54 shows three universal pulleys 316 installed as sliding devices on the bottom of the front base 310 and rear base 320 of the combination C 300.

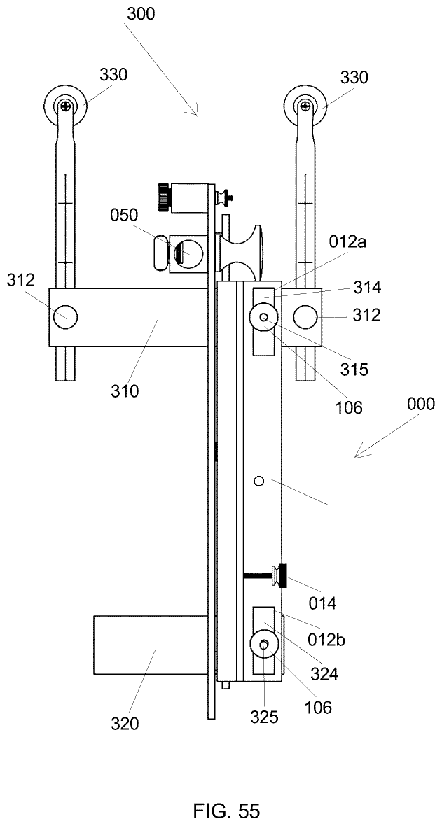

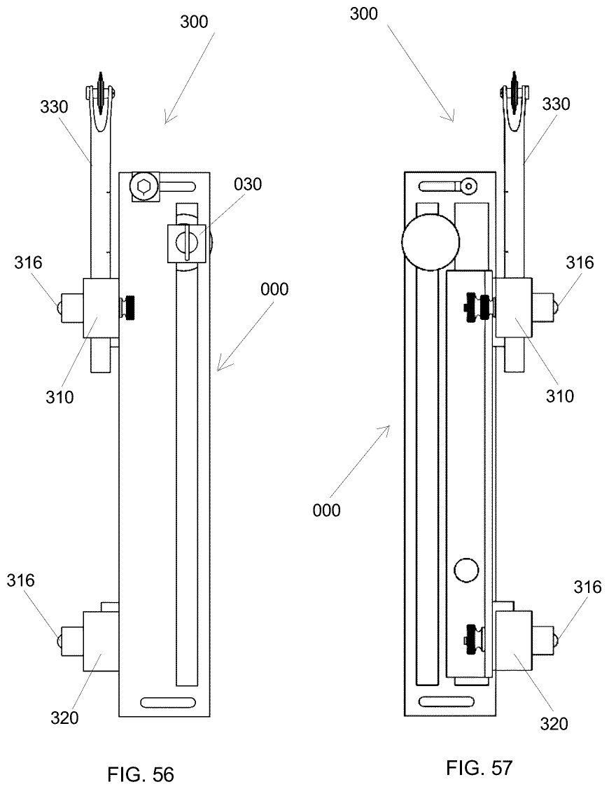

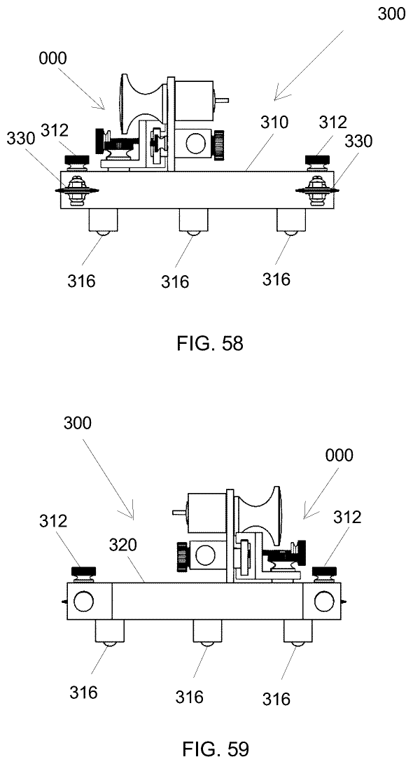

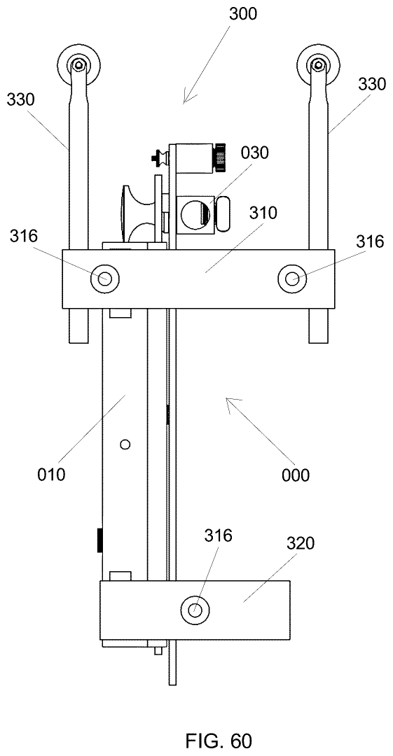

FIG. 55 is a top view of combination C 300, showing the main board 001 and front base 310 are perpendicular to each other through the joining of the rectangular blocks of the front and rear base 314 and 324 respectively to the rectangular holes 012a and 012b of the sliding guide bracket. The dual detector wheels 330 are adjusted to the same length and fixed with screws 312, so that the marker holder 030 is located centrally and equidistantly between the two detection wheels. The distance between the marker and the two detector wheels is symmetrical and equal, with the tactile sensation of the two detection wheels felt equally, regardless of marker location on the main board 001.

Continuing with the description of FIG. 55, the dual detector wheels 330, equidistant from the marker 050 of the combined C300 device contacts the surface of the object being measured and roll simultaneously, so that the marker pen 050 avoids displacement deviation and stably traces a contour.

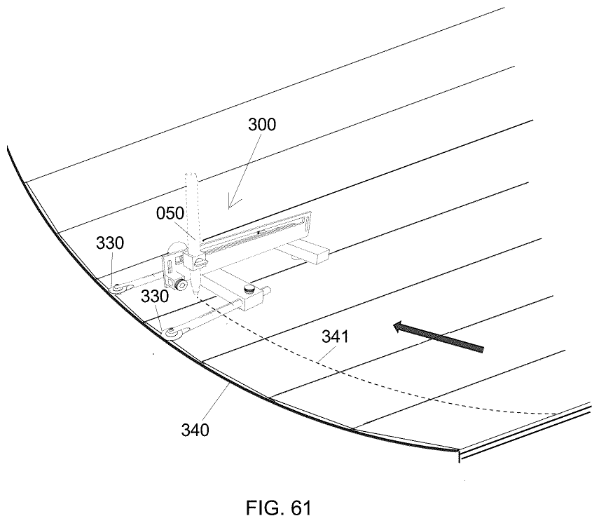

FIG. 61 is a reference implementation diagram of the measurement of a curved wall by Combination C 300. The Combination C 300 is held by hand (not shown) to allow the dual detector wheels 330 to contact and roll along the surface of wall 340. The marker 050 simultaneously records the trajectory 341 of the dual detector wheel movement on the wood flooring.

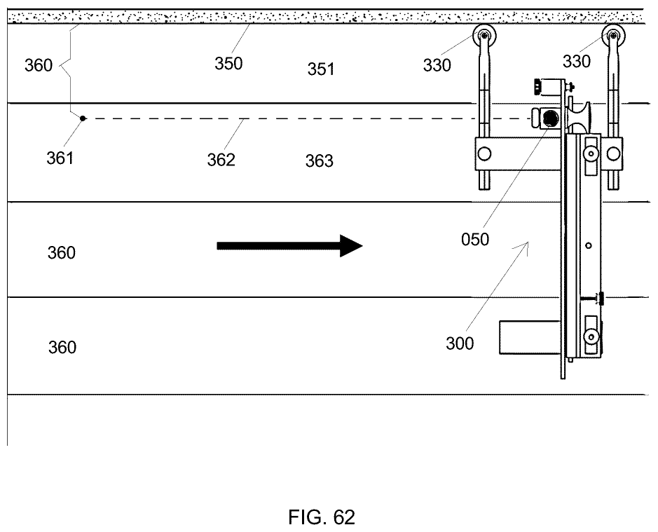

FIG. 62 is an implementation reference diagram of the combination C 300 measuring a flat wall covering space 351. The distance from wall 350 to the recording start point 361 is measured using the width of the wood flooring as the standard size. When the measurement is completed, marker 050 records track 362 of the double detector wheels 330 moving on the wall surface 350 on the wood flooring. Track 362 is a contour line parallel to wall 360. The dimensions of the flooring 363 below track 362 are the same as those in space 351.

Continuing with the description of FIG. 62, wood flooring 360 is removed and discarding the portion above the contour line 362. A complete wood flooring is re-laid at the original position, and then the cut wood flooring 363 is placed to cover the space 351.

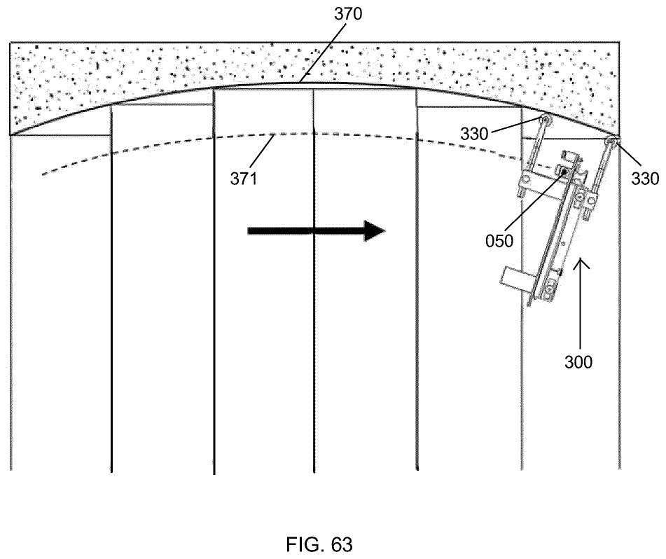

FIG. 63 is an implementation reference diagram of the combination C 300 measuring the inner curved surface of the wall. As the double detector wheel 330 rolls on the surface of wall 370, the marker 050 traces the contour 371 with the same curvature as the curved wall surface.

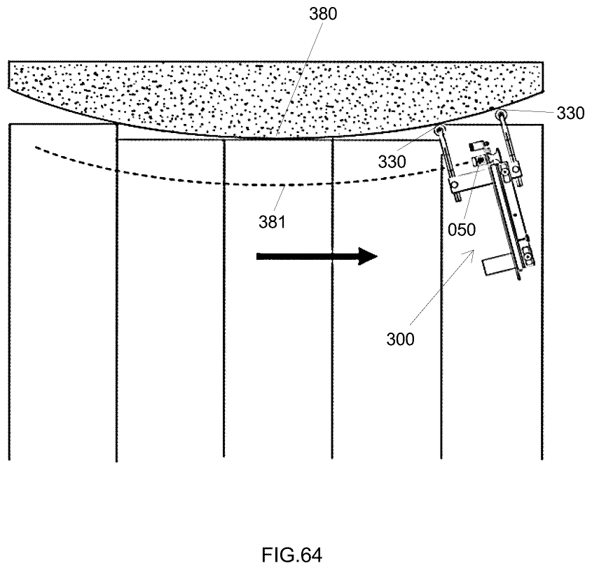

FIG. 64 is an implementation reference diagram of the combination C 300 that measures the outer curved surface of a wall. As the double detector wheel 330 rolls on the surface of the wall 380, the marker 050 records the contour 381 with the same curvature as the curved surface of the wall on the wood flooring.

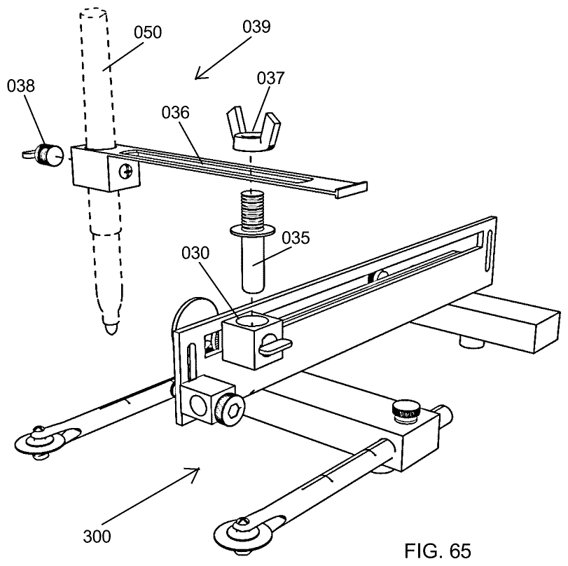

FIG. 65 is an exploded perspective view of Combination C equipped with an extended marker pen holder, showing the plunger screw 035, the marker pen holder extender 036, the butterfly nut 037 and the marker pen fixing screw 038. The extended marker pen holder is designated as 039.

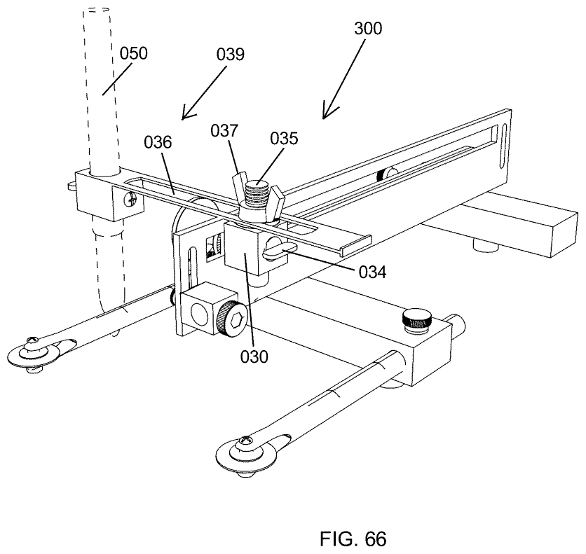

FIG. 66 is a combination C 300 equipped with an extended marker pen holder 039, wherein the plunger screw 035 is inserted into the marker pen holder 030 and secured by the screw 034, the narrow slot 036 of the marker pen holder extender is moved to a specific position within the physical slot of the plunger screw 035 and fixed with a butterfly nut 037. The marker pen 050 is then fixed in the round hole of the extended marker pen holder with a screw 038.

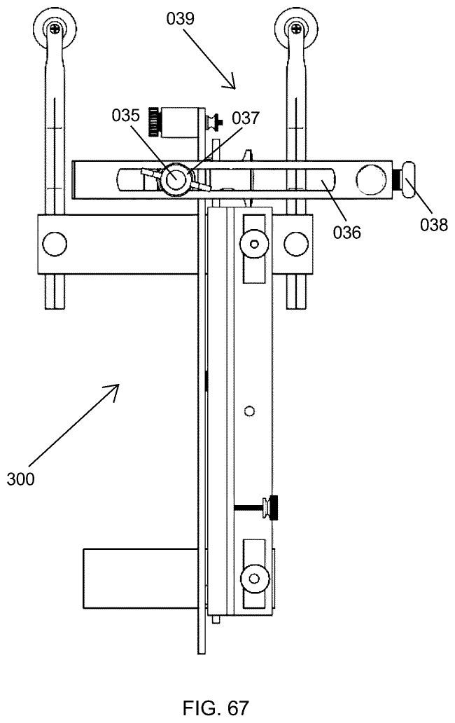

FIG. 67 shows a top view of the C 300 combination with the extended marker holder 039. Since the marker holder 030 is in the middle between the two detector wheels 330, the start and end of the contour cannot be recorded during the measurement. After the first measurement, the C 300 combination is placed at the start or end of the measurement with the extended marker holder 039 installed on the left or right. The missing lines are traced accordingly in addition to the already recorded contour.

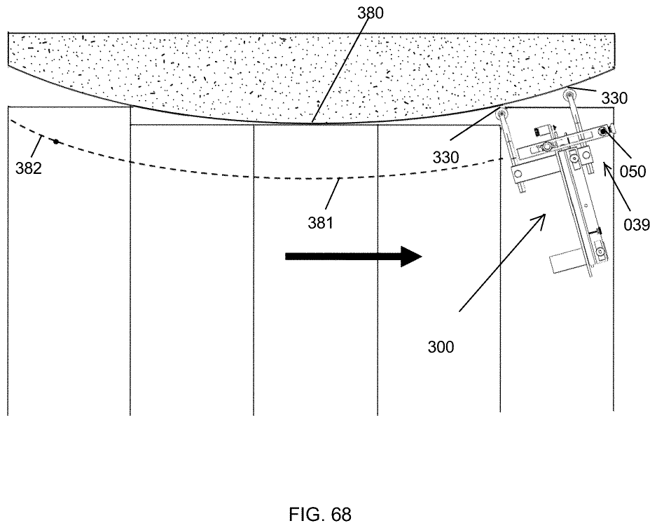

FIG. 68 is an implementation reference diagram of the combination C 300 equipped with an extended marker pen holder 039, in which the contour line 382 of the starting part is additionally recorded on the wood flooring 380, and the contour line of the ending part is being recorded.

The inventor(s) claim:

1. The multi-functional combination outline tool detects the shape of an object's surface and records its contour on a planar material, with the detection recorder as the main component and three different bases that can form three functionally unique tools, including A: the first combination outline tool, B: the second combination outline tool, and C: the third combination outline tool.

2. The multi-functional combination outline tool of claim 1, wherein said detection recorder comprises a main board, a detector holder, detectors, a marker pen holder, and a slide guide bracket.

3. The detection recorder of claim 2, wherein said main board comprises of a horizontal groove.

4. The detection recorder of claim 2, wherein said main board comprises of a slide bar.

5. The detection recorder of claim 2, wherein said main board comprises of vertical slots at each end.

6. The detection recorder of claim 2, wherein said detector holder comprises of a circular hole for mounting the detector and fastening screws, secured to the main board by a screw and fixing nut.

7. The detection recorder of claim 2, wherein said detectors comprises of a detector rod.

8. The detection recorder of claim 2, wherein said detectors comprises of a detector wheel.

9. The detection recorder of claim 2, wherein said marker pen holder comprises of a marker loading hole and a fastening screw, with a side convex width limiting to lateral movement, that secured on the main board with a screw and knob.

10. The detection recorder of claim 2, wherein said slide guide bracket comprises of a slide groove for lateral movement of the main board.

11. The detection recorder of claim 2, wherein said slide guide bracket comprises of rectangular holes at each end, with screw holes and fastening screws on the side of said slide guide bracket to secure to the main board.

12. The first combination outline tool of claim 1, wherein said front base comprises of rectangular blocks at each end that are perpendicular to the vertical edge.

13. The first combination outline tool of claim 1, wherein said front base comprises an operating handle.

14. The first combination outline tool of claim 1, wherein said front base comprises of the vertical edge of front base.

15. The second combination outline tool of claim 1, wherein said rectangular base is comprised of a detection recorder, a slide guide bracket, a front base, a rear base, and a combined connecting link.

16. The rectangular base of claim 15, wherein said clamp is formed by the vertical edges of the front base and the rear base.

17. The rectangular base of claim 15, wherein said combined connecting link is comprised of a grooved connecting link and grooved sub-connecting link.

18. The third combination outline tool of claim 1, wherein said tool comprises of front base, rear base, and dual detection wheels.

19. The third combination outline tool of claim 18, wherein said front base comprises of a rectangular block perpendicular to said front base.

20. The third combination outline tool of claim 18, wherein said front base comprises of detector wheel mounting holes on the vertical sides that pass through the width at each end of the said front base.

21. The third combination outline tool of claim 18, wherein said front base comprises of screw holes and fastening screw located perpendicularly above each mounting hole.

22. The third combination outline tool of claim 18, wherein said tool comprises of dual detector wheels secured to the front base.

23. The third combination outline tool of claim 18, wherein said rear base comprises of a rectangular block perpendicular to the said rear base.

24. The third combination outline tool of claim 18, wherein said sliding device comprises of two universal pulleys on the bottom of the front base and one universal pulley on the bottom of the rear base.

25. The third combination outline tool of claim 18, wherein said marker pen holder is of extended form.

26. A method for measuring the contour of an object using the first combination outline tool clamped on the vertical edge of the wood flooring, or flat material, as a basis of reference, comprising of the following: moving the vertical edge of the front base laterally along the edge of the wood flooring, the detector rod (or wheel) of the detection recorder detects the change in object shape, the marker simultaneously records the trajectories' lateral and longitudinal movement on the wood flooring.

27. A bisectional method for measuring the complete contour of an object using the second combination outline tool, comprising of the following steps: determine the center points of both ends of the object, align the two ends of the wood flooring and place them parallel to the central axis formed by the center points on both ends and sides of the object, clamp the rectangular base to the wood flooring and have the tip of the detector rod contact a center point at one end of the object, have the marker begin recording at the edge of the wood flooring, move the detector rod along the edge until reaching the second center point, the marker would have synchronously recorded the half of the object's contour, repeat the operation for the second half of the object's contour, align and merge the two wood floorings to obtain the contour of the overall object.

28. A method for measuring the contour of a vertical plane or the curved surface of an object with the third combination outline tool, using dual detection wheels to trace a line according to the said object's surface. First, the dual detection wheels are adjusted to identical lengths and fixed, with a marker adjusted to the preferred position. The dual detection wheels are to contact and roll across the surface of the object for the marker to record the moving trajectory of the said object's surface. The resultant line recorded by the dual detection wheels on the wood flooring will have the same trajectory and curvature as the surface of the object.
